(19) United States
(12) Patent Application Publication
(10) Pub. No.: US 2007/0167745 A1
(43) Pub. Date: Jul. 19, 2007

(54) METHODS FOR DELIVERING MEDICAL DEVICES TO A TARGET IMPLANT SITE WITHIN A BODY VESSEL

(75) Inventor: Brian C. Case, Lake Villa, IL (US)

Correspondence Address: DUNLAP, CODDING & ROGERS, P.C., P.O. BOX 16370, OKLAHOMA CITY, OK, 73113, US

(73) Assignee: COOK INCORPORATED, BLOOMINGTON, IN (US)

(21) Appl. No.: 11/617,846

(22) Filed: Dec. 29, 2006

Related U.S. Application Data
(60) Provisional application No. 60/754,784, filed on Dec. 29, 2005.

(51) Int. Cl. A61B 5/05

(52) U.S. Cl. 600/424

(57) ABSTRACT

Methods of delivering a medical device to a point of treatment in a body vessel are described. In methods according to the invention, a reference device comprising a plurality of reference markings and a medical device comprising at least one position marker are provided. The reference device is associated with an external surface of the patient in a region adjacent the body vessel in which the medical device is to be implanted. A target implant site is determined and correlated with at least one selected reference marking on the reference device. The medical device is percutaneously advanced to the target implant site by visually referencing the at least one selected reference marking and the at least on position marker using an appropriate imaging system.

### CROSS-REFERENCE TO RELATED APPLICATIONS

This application claims priority to United States Provisional Application Ser. No. 60/754,784, filed on Dec. 29, 2005, the entire disclosure of which is hereby incorporated into this disclosure.

### FIELD OF THE INVENTION

The present invention relates to methods for delivering medical devices to a point of treatment in a body vessel. Exemplary methods according to the invention relate to methods for delivering a medical device to modify venous blood flow in a patient, such as a valve device. Methods according to exemplary embodiments are particularly useful in the treatment of venous insufficiency and other diseases and conditions of the vasculature.

### BACKGROUND

Minimally invasive techniques and instruments for placement of intraluminal medical devices have been developed over recent years and are frequently used to deliver an intraluminal medical device to a desired point of treatment and deploy the intraluminal medical device at the point of treatment. In these techniques, a delivery system is used to carry the intraluminal medical device through a body vessel and to the point of treatment. Once the point of treatment is reached, the intraluminal medical device is deployed from the delivery system for implantation. The delivery system is subsequently withdrawn from the point of treatment and, ultimately, the body vessel. A wide variety of treatment devices that utilize minimally invasive technology have been developed, including stents, stent grafts, occlusion devices, infusion catheters, prosthetic valves, and the like.

Prosthetic valves for modifying blood flow in body vessels can be delivered to a point of treatment using minimally invasive techniques. In these procedures, the prosthetic valve is delivered to a target implant site within a body vessel, frequently a vein in the leg of a patient. Imaging equipment and techniques can be used to visualize the prosthetic valve before, during, and after deployment.

It is desirable in many procedures to deploy prosthetic valves at a predetermined distance from a point of interest, such as an anatomical landmark. Accordingly, a need exists for medical devices that facilitate placement of an intraluminal medical device at a desired point of treatment. There is a need for delivery systems that offer more accurate delivery of medical devices near the desired point of treatment. As prosthetic valves and their use to modify fluid flow in body vessels is gaining acceptance, there is a need for new and useful methods for delivering these medical devices to points of treatment within body vessels.

### SUMMARY OF EXEMPLARY EMBODIMENTS

The invention provides methods for delivering medical devices to modify venous blood flow in a patient. An exemplary method according to the invention comprises the steps of providing an imaging system; associating a reference device that includes a plurality of reference markings visible with the imaging system with an exterior surface of a patient in a region in which a prosthetic valve is to be implanted; determining a target implant site in a body vessel within the region in which a prosthetic valve is to be implanted; correlating the target implant site with at least one reference marking of the reference device; providing a prosthetic valve having at least one position marker that is visible with the imaging system; percutaneously advancing the prosthetic valve to the target implant site by visually referencing the at least one reference marking and the at least one position marking.

Methods according to the invention can optionally include a step of determining a valve location of at least one insufficient valve in the body vessel. In these methods, a target implant site can be determined relative to the valve location. One exemplary method includes the step of percutaneously delivering into a body vessel a contrast agent visible with the imaging system under conditions effective to identify a valve location of at least one insufficient valve in the body vessel. Other techniques and apparatuses can be used in the step of determining a valve location of at least one insufficient valve. For example, an endoluminal device that facilitates visualization of valves from within the body vessel, such as an intravascular ultrasound (IVUS) device, can be used.

The invention includes other embodiments within the scope of the claims, and variations of all embodiments. Additional understanding of the invention can be obtained by referencing the detailed description of exemplary embodiments of the invention, below, and the appended drawings.

### BRIEF DESCRIPTION OF THE DRAWINGS

FIG. 1 is a flow diagram of a method according to a first exemplary embodiment of the invention.

FIG. 2 is a schematic of a patient on which a method according to the invention is being performed.

FIG. 3 is a sectional view of a region of a patient on which a method according to the invention is being performed.

FIG. 4 is a flow diagram of a method according to a second exemplary embodiment of the invention.

### DETAILED DESCRIPTION OF EXEMPLARY EMBODIMENTS

The following detailed description and appended drawings describe and illustrate various exemplary embodiments of the invention. The description and drawings serve to enable one skilled in the art to practice the invention. They are not intended to limit the scope of the invention, or its protection, in any manner.

The invention provides methods for delivering a medical device to modify fluid flow in a body vessel of a patient. Methods of the invention are useful in the treatment and/or diagnosis of a variety of medical conditions, including venous insufficiency and other pathologies of the veins, arteries, and other body vessels.

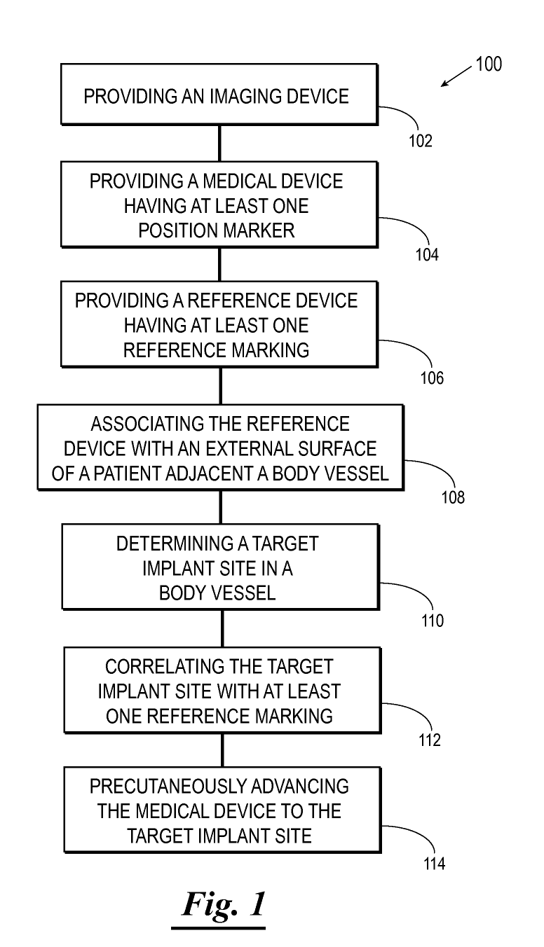

An exemplary method 100 according to the invention is represented schematically by the flowchart illustrated in FIG. 1. The method 100 comprises a step 102 of providing an imaging system; another step 104 of providing a medical device that comprises at least one position marker that is visible with the imaging system; another step 106 of providing a reference device that includes a plurality of reference markings; another step 108 of associating the reference device with an external surface of a patient in a region adjacent the body vessel in which the medical device is to be implanted; another step 110 of determining a target implant site in the body vessel; another step 112 of correlating the target implant site with at least one selected reference marking of the plurality of reference markings that are associated with the reference device; and another step 114 of percutaneously advancing the medical device to the target implant site by visually referencing the at least one selected reference marking and the at least one position marker of the medical device with the imaging system.

Step 102 comprises providing an imaging system. Any suitable imaging system can be used in the practice of the invention. The imaging system selected need only be compatible with the plurality of reference markings associated with the reference device and the at least one position marker associated with the medical device. The particular imaging system selected for a specific method according to the invention will depend on several considerations, including the nature of the medical device and the body vessel in which the device will be implanted. Those skilled in the art will be able to determine a suitable imaging system for use in a specific method according to the invention. Examples of suitable imaging systems include computer tomography systems, magnetic resonance imaging systems, ultrasound systems, and fluoroscopy systems.

The inventors have determined that methods according to the invention are particularly useful in the implantation of medical devices for the treatment of venous insufficiency and other conditions affecting the vasculature. In these methods, ultrasound imaging systems typically used in venography techniques and procedures are suitable.

As described more fully below, both the plurality of reference markings associated with the reference device and the at least one position marker associated with the medical device need to be compatible with the imaging system. Accordingly, appropriate reference markings and position markers (and reference devices and medical devices) can be selected based upon the type of imaging system employed. Those skilled in the art can determine suitable markings and markers for use with particular imaging systems.

Step 104 comprises providing a medical device that comprises at least one position marker that is visible with the imaging system. Any suitable intraluminal medical device can be used with the methods according to the invention. Examples of suitable intraluminal medical devices include stents, filters, embolization devices, and valve devices. Both self-expandable and balloon-expandable intraluminal medical devices can be used.

The inventors have determined that methods according to the invention are particularly well-suited for use in the treatment and/or diagnosis of venous insufficiency. Accordingly, intraluminal medical devices well-suited for the treatment and/or diagnosis of this condition are particularly well-suited for use in methods according to the invention. Self-expandable valves provide an example of a suitable intraluminal medical device. Examples of self-expandable valves include those described in U.S. Pat. No. 6,200,336 to Pavcnik et al. for a MULTIPLE-SIDED INTRALUMINAL MEDICAL DEVICE; U.S. application for patent Ser. No. 10/642,372 of Pavcnik et al. for an IMPLANTABLE VASCULAR DEVICE, filed on Aug. 15, 2003; and U.S. application for patent Ser. No. 10/828,716 of Case et al. for an ARTIFICIAL VALVE PROSTHESIS WITH IMPROVED FLOW DYNAMICS, filed on Apr. 21, 2004; the entire disclosures of which are hereby incorporated into this disclosure for the purpose of describing suitable intraluminal medical devices for use in methods according to the invention.

Step 106 comprises providing a reference device that includes a plurality of reference markings. The reference device includes a plurality of reference markings that are compatible with the imaging system used in a specific method according to the invention. Any suitable set of reference markings can be used as the plurality of reference markings. A scale that includes reference markings spaced at regular intervals is considered advantageous at least because such an arrangement facilitates the step of correlating a target implant site with at least one of the reference markings. A scale with regular spacing is also considered advantageous because is provides a standard set of distances on which users can rely for spacing determinations, which may be useful if a medical device is being implanted at a predetermined distance from a target site, as described more fully below. The particular scale used in a specific reference device will depend on various considerations, including the nature of the body vessel, medical device, and condition being treated and/or diagnosed. Those skilled in the art can determine a suitable scale for use in specific methods according to the invention.

The reference device is a device intended for use on or with an external surface of the patient. The device advantageously includes a means for temporarily attaching the device to an external surface of a patient. Any suitable means for attaching a device to an external surface of a patient can be used, and suitable examples include adhesives and attachment elements such as clips.

The inventors have determined that an elongate member including an adhesive surface on one side and a plurality of reference markings on an opposing side is a suitable reference device for use in the methods according to the invention. The elongate member is advantageously a flexible member and capable of rolling onto itself to facilitate storage. Such a reference device is considered advantageous at least because of its ease of use and its ability to be attached temporarily to an external surface of a patient with relative ease.

A phosphorescent, fluorescent, or other luminescentt material may be added to the reference device and/or reference markings making the reference device and/or reference markings ‘glow in the dark.’ Such a reference device is considered advantageous in ‘low-light’ procedures such that in conditions with limited external light, the ‘glow in the dark’ material aids in making the reference markings visiable.

A pattern temporarily projected onto an external surface of the patient, such as by light passed through a template defining the pattern, is another example of a suitable reference device. This type of reference device is considered advantageous at least because it minimizes waste associated with practicing the invention and eliminates the need to attach the reference device to the external surface of the patient. When such a reference device is used, the step of associating a reference device with an external surface of the patient simply comprises the act of projecting the pattern onto the surface of the patient. The reference device can be removed at an appropriate time by terminating the projection onto the surface of the patient.

Step 108 comprises associating the reference device with an external surface of a patient in a region adjacent the body vessel in which the medical device is to be implanted. As used herein in relation to this step, the term “associating” refers to temporarily positioning the reference device in a region adjacent the body vessel in which the medical device is to be implanted. The term does not require a permanent attachment to a body surface, nor does it require attachment at all. The reference device need only be positioned near the body vessel in such a manner that allows the one or more of the reference markings to be visually referenced with the imaging system.

Step 110 comprises determining a target implant site in the body vessel. The target implant site can comprise any suitable site within the body vessel and the particular implant site chosen for a specific method according to the invention will depend on several considerations, including the nature of the body vessel, the nature of the medical device, and the condition being treated and/or diagnosed. Those skilled in the art will be able to determine an appropriate target implant site based on these and potentially other considerations.

A target implant site can be determined using a variety of techniques. For example, in the treatment of venous insufficiency, as described more fully below, the location of a natural venous valve can be determined using suitable equipment and techniques. A target implant site can then be determined either as the location of the natural valve or a site spaced from the natural valve by a predetermined distance.

Alternatively, an anatomical point of reference can be identified, such as a palpable point of reference or a visual point of reference. A target implant site can then be determined based upon a relationship to the anatomical point of reference. The medical device can be percutaneously advanced to the target implant site by referencing one or more reference markers of the reference device that correlate with the anatomical point of reference.

Step 112 comprises correlating the target implant site with at least one selected reference marking of the plurality of reference markings that are associated with the reference device. This is accomplished using the imaging system to visualize the plurality of reference markings and selecting one reference marking that correlates to the target implant site. For example, a reference marking that lies directly adjacent the target implant site can be selected from the plurality of reference markings.

Step 114 comprises percutaneously advancing the medical device to the target implant site by visually referencing the at least one selected reference marking and the at least one position marker of the medical device with the imaging system. The medical device can be percutaneously advanced to the target implant site using minimally invasive techniques and devices, such as catheter-based delivery systems and wireguides known to those skilled in the art. During at least a portion of the percutaneous advancement of the medical device, the user visually references the selected reference marking, which correlates to the target implant site, and the position marker of the medical device using the imaging system. Once the position marker is positioned sufficiently near or directly adjacent the selected reference marking, the user knows that the medical device has reached the target implant site and can then deploy the medical device using techniques suitable for the minimally invasive devices being used. For example, the user may withdraw a sheath member associated with a catheter-based delivery device to expose and deploy a self-expanding medical device.

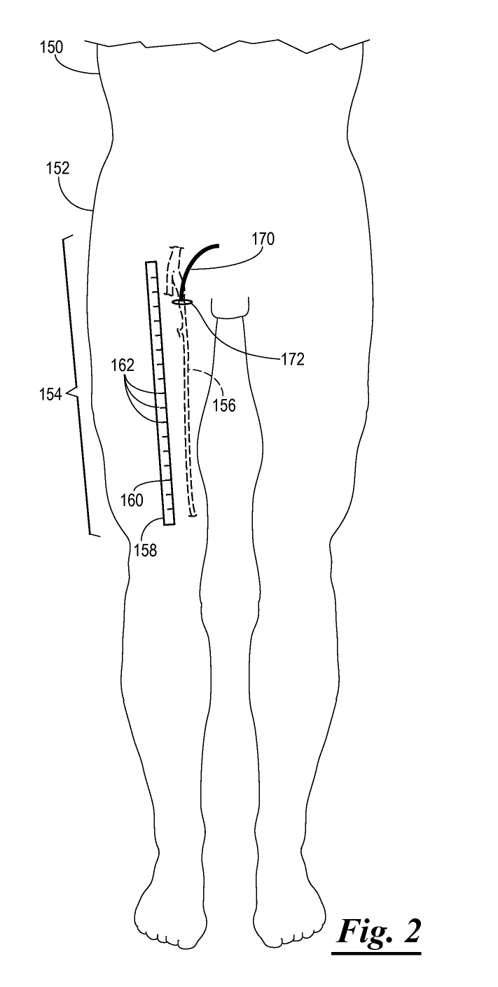

FIG. 2 schematically illustrates a patient 150 on which a method according to the invention is being performed. A leg 152 of the patient includes a region 154 in which a body vessel 156 is contained. A reference device 158 is secured to the skin of the patient in the region 154 and adjacent the body vessel 156. The reference device 158 comprises an elongate member and includes a scale 160 comprising a plurality of reference markings 162. The illustrated reference device is adhesively secured to the skin of the patient 150.

A wireguide 170 has been placed partially within the body vessel 156 through an access site 172. The wireguide 170 defines a path along which a medical device can be advanced to a target implant site within the body vessel 156, such as by advancing a catheter-based delivery system along the wireguide 170.

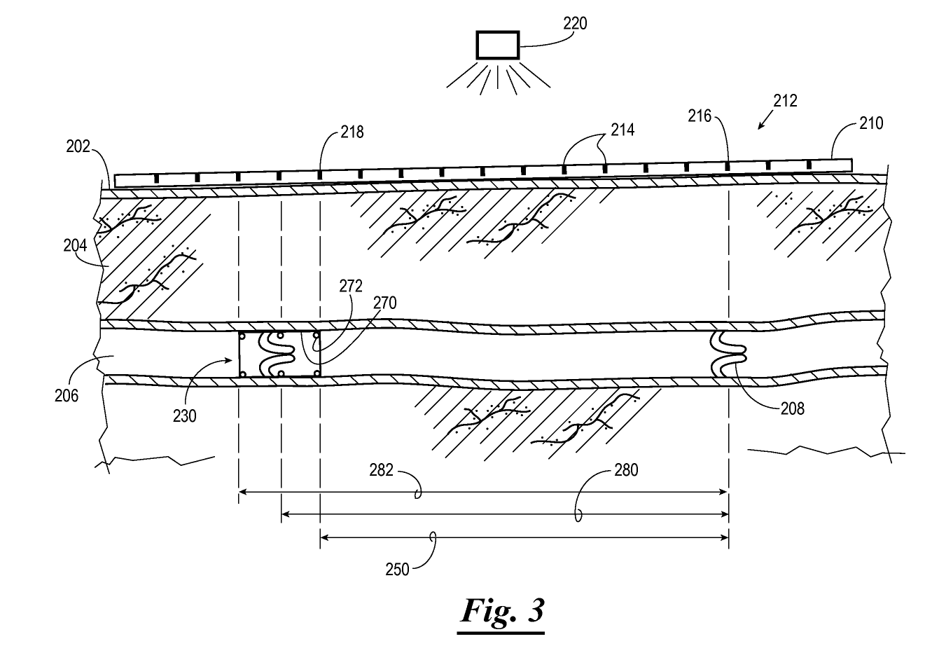

FIG. 3 schematically illustrates a cross-section of a region 200 of a patient on which a method according to the invention is being performed. In the sectional view, the external surface 202 comprises the skin of the patient. Various tissue 204 underlies the external surface 202 and a body vessel 206 is disposed within the tissue 204. The body vessel 206 includes a natural valve 208.

A reference device 210 is attached to the external surface 202 and includes a scale 212 comprising a plurality of reference markings 214.

An imaging system 220 is disposed near the body vessel 206 and the attached reference device 210.

A target implant site 230 is identified based upon a predetermined distance 250 between a first reference marking 216 of the reference device 210 and a second reference marking 218 of the reference device.

A medical device 270 is positioned within the body vessel 206 at the target implant site 230. The medical device 270 has been placed at the target implant site 230 by visually referencing the second reference marking 218 and at least one position marker 272 associated with the medical device. One or more additional predetermined distances, such as 280, 282, can be used to identify the target implant site and/or to facilitate positioning of the medical device 270 at the target implant site.

Methods according to the invention can optionally include additional and alternative steps. For example, the inventors have determined that in some methods according to the invention, it may be advantageous to determine the location of an insufficient valve within a body vessel prior to implantation of the medical device. Such a determination can be used to identify a target implant site based upon the location of the insufficient valve. For example, it may be advantageous to define a target implant site that is substantially the same axial location of the insufficient valve in the body vessel. This facilitates placement of medical devices at the location of the insufficient valve within the body vessel, which may be desirable with certain types of medical devices used in the treatment of venous insufficiency. Alternatively, a target implant site can be identified by defining a location that is axially spaced from the location of the insufficient valve by a predetermined distance. This facilitates placement of medical devices at a location that is distinct from the location of the insufficient valve, which may be desirable with certain types of medical devices used in the treatment of venous insufficiency.

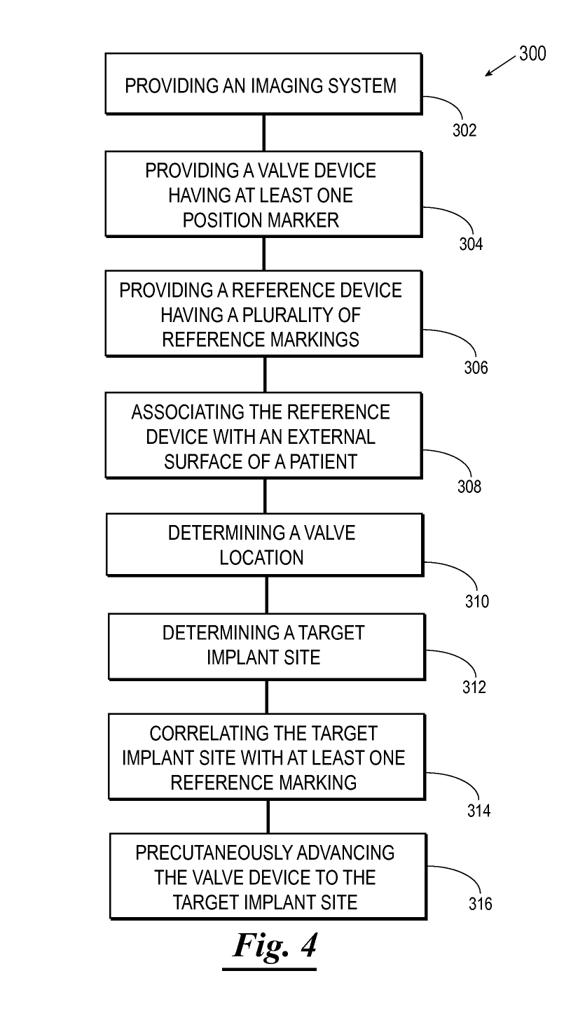

FIG. 4 schematically illustrates an exemplary method 300 according to the invention. The method 300 comprises a step 302 of providing an imaging system; another step 304 of providing a valve device that comprises at least one position marker that is visible with the imaging system; another step 306 of providing a reference device that includes a plurality of reference markings; another step 308 of associating the reference device with an external surface of a patient in a region adjacent the body vessel in which the medical device is to be implanted; another step 310 of determining a valve location of at least one insufficient valve in the body vessel; another step 312 of determining a target implant site in the body vessel; another step 314 of correlating the target implant site with at least one selected reference marking of the plurality of reference markings that are associated with the reference device; and another step 316 of percutaneously advancing the valve device to the target implant site by visually referencing the at least one selected reference marking and the at least one position marker of the medical device with the imaging system.

If the optional step of determining the location of an insufficient valve within a body vessel prior to implantation of the medical device is included, any suitable technique and equipment can be used to determine the location of an insufficient valve. The inventors have determined that a step of percutaneously delivering a contrast agent that is visible with the imaging system into the body vessel is particularly advantageous because it allows for easy visual identification of a valve location using the imaging system already being employed in the method. Alternatively, an endoluminal device that includes a position indicating marker that is visible with the imaging system and that is able to detect the location of a valve from within the body vessel can be used. For example, an intravenous ultrasound (IVUS) catheter that includes a position indicating marker visible with the imaging system can be used. The IVUS catheter can be used to visually identify a valve from within the body vessel. Once the valve is identified by IVUS, the position indicating marker can be visualized using the imaging system and correlated to a reference marking on the reference device.

Also alternatively, the step of correlating the target implant site with at least one reference marking can include the use of a second reference marking of the plurality of reference markings on the reference device. For example, the valve location can be correlated with a second reference marking. A reference marking for correlation with the target implant site can be determined by determining which reference marking of the plurality of reference markings is spaced from the second reference marking by a desired predetermined distance. Once the reference marking is determined, the target site can be correlated with that reference marking, and the medical device can be percutaneously advanced to the target implant site by visually referencing the determined reference marking and a position marker associated with the medical device.

The foregoing detailed description provides exemplary embodiments of the invention and includes the best mode for practicing the invention. The description and illustration of embodiments are intended only to provide examples of the invention; they are not intended to limit the scope of the invention, or its protection, in any manner.

What is claimed is:

1. A method for delivering a medical device to a point of treatment in a body vessel of a patient, comprising:
providing an imaging system;
providing a medical device comprising at least one position marker visible with the imaging system;
providing a reference device comprising a plurality of reference markings;
associating the reference device with an external surface of said patient in a region adjacent said body vessel;
determining a target implant site in said body vessel;
correlating the target implant site with at least one selected reference marking of the plurality of reference markings; and
percutaneously advancing the medical device to the target implant site by visually referencing the at least one selected reference marking and the at least one position marker with the imaging system.

2. A method for delivering a medical device according to claim 1, wherein the imaging system is one of a computer tomography system, a magnetic resonance imaging system, an ultrasound imaging system, and a fluoroscopy imaging system.

3. A method for delivering a medical device according to claim 1, wherein the reference device comprises an adhesive surface.

4. A method for delivering a medical device according to claim 3, wherein the step of associating a reference device comprises contacting the adhesive surface to said patient.

5. A method for delivering a medical device according to claim 1, further comprising the step of identifying a valve location of at least one insufficient valve in said body vessel.

6. A method for delivering a medical device according to claim 5, wherein the step of identifying a valve location of at least one insufficient valve comprises percutaneously delivering into said vein a contrast agent visible with the imaging system.

7. A method for delivering a medical device according to claim 5, wherein the step of identifying a valve location of at least one insufficient valve comprises percutaneously advancing into said body vessel an endoluminal device comprising a position indicating marker visible with the imaging system.

8. A method for delivering a medical device according to claim 7, wherein the endoluminal device comprises an ultrasound transducer.

9. A method for delivering a medical device according to claim 5, wherein the target implant site is axially spaced from the valve location by a predetermined distance.

10. A method for delivering a medical device according to claim 9, wherein the predetermined distance is substantially the same as a distance between proximal and distal reference markings of the plurality of reference markings.

11. A method for delivering a medical device according to claim 1, wherein the medical device comprises a prosthetic valve.

12. A method for delivering a medical device to modify fluid flow in a body vessel of a patient, comprising:
providing an imaging system;
providing a valve device comprising at least one position marker visible with the imaging system;
providing a reference device comprising a plurality of reference markings;
associating the reference device with an external surface of said patient in a region in which the prosthetic valve is to be implanted;
determining a valve location of at least one insufficient valve in said body vessel;
determining a target implant site in said body vessel relative to the valve location;
correlating the target implant site with at least one reference marking of the plurality of reference markings; and
percutaneously advancing the valve device to the target implant site by visually referencing the at least one reference marking and the at least one position marker with the imaging system.

13. A method for delivering a medical device according to claim 12, wherein the imaging system is one of a computer tomography system, a magnetic resonance imaging system, an ultrasound imaging system, and a fluoroscopy imaging system.

14. A method for delivering a medical device according to claim 12, wherein the reference device comprises an adhesive surface.

15. A method for delivering a medical device according to claim 14, wherein the step of associating a reference device comprises contacting the adhesive surface to said patient.

16. A method for delivering a medical device according to claim 12, wherein the target implant site is substantially the same as the valve location.

17. A method for delivering a medical device according to claim 12, wherein the target implant site is axially spaced from the valve location by a predetermined distance.

18. A method for delivering a medical device according to claim 12, further comprising correlating the valve location with a second reference marking of the plurality of reference markings.

19. A method for delivering a medical device according to claim 18, wherein the step of correlating the target implant site with at least one reference marking of the plurality of reference markings comprises determining the at least one reference marking of the plurality of reference markings based upon a predetermined interval of the plurality of reference markings relative to the second at least one reference marking of the plurality of reference markings.

20. A method for delivering a medical device in a body vessel of a patient; comprising:
providing an imaging system;
providing a valve device comprising at least one position marker visible with the imaging system;
associating a reference device with an external surface of said patient in a region in which the prosthetic valve is to be implanted, the reference device including a plurality of reference markings visible with the imaging system;
determining an anatomical point of reference;
correlating the anatomical point of reference with a first reference marking of the plurality of reference markings; and
percutaneously advancing the valve device to a target implant site by visually referencing a second reference marking and the at least one position marker with the imaging system, the second reference marking spaced from the first reference marking by a predetermined distance.
